(19) United States
(12) Patent Application Publication
(10) Pub. No.: US 2003/0135394 A1
(43) Pub. Date: Jul. 17, 2003

(54) Health services delivery system with incentives

(76) Inventors: Nicanor Padron, Miami, FL (US); Jose Julian Padron, Miami, FL (US)

Correspondence Address: MALLOY & MALLOY, P.A., 2800 S.W. Third Avenue, Historic Coral Way, Miami, FL, 33129, US

(21) Appl. No.: 10/340,929

(22) Filed: Jan. 13, 2003

Related U.S. Application Data
(63) Continuation-in-part of application No. 09/603,529, filed on Jun. 23, 2000, now abandoned.

(51) Int. Cl. G06F 17/60; A61B 5/00

(52) U.S. Cl. 705/3; 705/2

(57) ABSTRACT

A system of health care service and an associated method of the administration thereof to a plurality of subscribers including a providing entity organizationally structured to implement a health care program which includes categorizing subscribers desirous of health care service, including preventative services, into a plurality of predetermined profiles. The profiles are defined by personal characteristics including age, gender, clinical history, etc. and each profile is associated with a predetermined curative or preventative treatment regimen with which a respective subscriber is expected to comply. An incentive system is implemented to motivate the subscribers into compliance with the assigned treatment regimen and comprises the assignment of credits and penalties based on a degree of compliance with the treatment regimen. Valuable consideration is awarded to the subscriber evidencing a satisfactory degree of compliance.

### CLAIM OF PRIORITY

The present application is a continuation-in-part application of previously filed, now pending application having Ser. No. 09/603,529 filed on Jun. 23, 2000.

### BACKGROUND OF THE INVENTION

1. Field of the Invention

The present invention is directed to a health care system and a method for the administration thereof by a providing entity, wherein a plurality of subscribers desirous of health care service are profiled according to personal characteristics and assigned a treatment regimen dependent on medical needs, preferably even before they exhibit a need for any health care services. A motivating incentive system is implemented to facilitate compliance with the assigned treatment regimen and involves the assignment of both credits and penalties to the subscriber based on the degree of compliance with the corresponding treatment regimen. Motivational awards are provided to the subscriber evidencing a satisfactory compliance with the assigned treatment regimen and ensuring in the best possible manner the effectiveness of preventative, family and community medicine plans.

2. Description of the Related Art

Many business and operational arrangements have been designed in the past for delivering health services to subscribers. Health maintenance organizations have been created with different philosophies to accomplish this at a minimum cost. However, systems that include incentives to their subscribers constitute a minority. None of them, however, include a system that issues credits as equity units in the use of the system when a subscriber complies with a sequence of events associated with the profile and/or treatment regimen in which he/she has been classified.

One of these incentive systems is described in U.S. Pat. No. 5,806,045 issued to Biorge et al. in 1998, and directed to a method and system for allocating and redeeming incentive credits between a portable device and a base device. This system is typical in that it accumulates credits that can be redeemed in subsequent transactions. However, the system and method of this patent is absent the classification of subscribers or users under a particular profile that requires them to perform certain acts for the incentives to be awarded, as it is the case in the present invention, as will be described hereinafter.

Applicant believes that another of these systems is disclosed in U.S. Pat. No. 5,301,105 issued to Cummings, Jr. in Apr. 5, 1994 for All Care Health Management System. The Cumming's system describe a fully integrated health care system and its interaction of the patient, health care provider, financially institution, etc. However, it differs from the present invention because it does not include a system that issues credits as equity units in the use of the system, and does not take effect immediately upon enrollment, but rather waits for an ailment requiring treatment to arise.

Other patents describing the closest subject matter provide for a number of more or less complicated features that fail to solve the problem in an efficient and economical way. None of these patents suggest the novel features of the present invention, which, among other benefits, involves all of the subscribers, in the system of incentives and penalties, and in addition to providing health benefits according to the subscriber's medical profile, provides a parallel for capitalization that is created with the economic funds assigned for the implementation of the incentives and penalties system, enabling in some instances a loan company to participate in the health services by mutual agreement and as permitted by law and other regulations.

### SUMMARY OF THE INVENTION

It is one of the main objects of the present invention to provide a system and method for providing and administering health services to a number of subscribers at minimum cost while enhancing their health.

It is another object of this invention to provide a system and method that permits the user to selectively provide incentives to those subscribers who participate in a number of pre-determined prescribed activities intended to improve their health or prevent more serious health consequences, without requiring the enrolled patient to suffer from a specific ailment in order to initiate the treatment.

It is still another object of the present invention to provide a system and method of implementation that is inexpensive and effective.

Further objects of the invention will be brought out in the following part of the specification, wherein detailed description is for the purpose of fully disclosing the invention without placing limitations thereon.

### BRIEF DESCRIPTION OF THE DRAWINGS

With the above and other related objects in view, the invention comprises details which will be more fully understood from the following description, when read in conjunction with the accompanying drawings in which:

FIG. 1 is a schematic representation of a preferred embodiment of a health care delivery system and method of the present invention.

FIG. 2 is a schematic representation in the form of a flow chart disclosing the steps of administering the health services to subscribers.

FIG. 2a is a flow chart disclosing operative features of a central processing unit included in the preferred embodiment of the present invention.

FIG. 3 is schematic representation of the preferred embodiment of the present invention disclosed in greater detail.

### DETAILED DESCRIPTION OF THE PREFERRED EMBODIMENT

As shown in the accompanied drawings, the health care delivery system and method of the present invention comprises a plurality of subscribers 20, desirous of receiving health care services, entering into a contractual obligation with a provider or providing entity 20 responsible for the performance and administration of the health care delivery system and method of the present invention. As such, the providing entity 20 has access at to and utilization of a processor and/or computerized assembly 30 with an appropriate data bank and associated memory 32 readily accessible for both the input and output of data as schematically indicated. In addition, the providing entity 20 may be associated with a plurality of vendors, normally comprising doctors or other health care professionals. These “outside” vendors are consulted in situations where a specialist and/or medical procedure is required for proper treatment of a subscriber 40, which may not be normally available on an “in-house” basis by the providing entity 20.

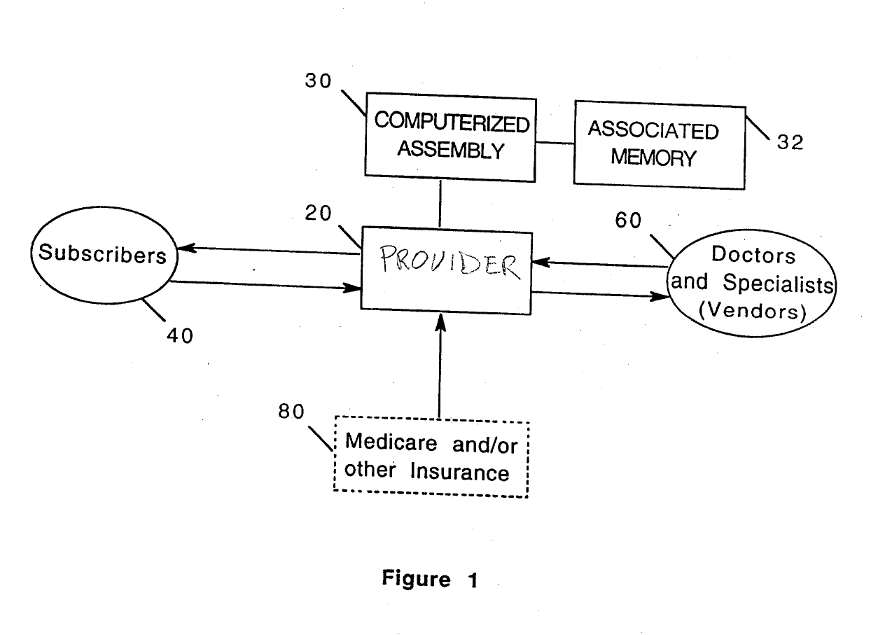

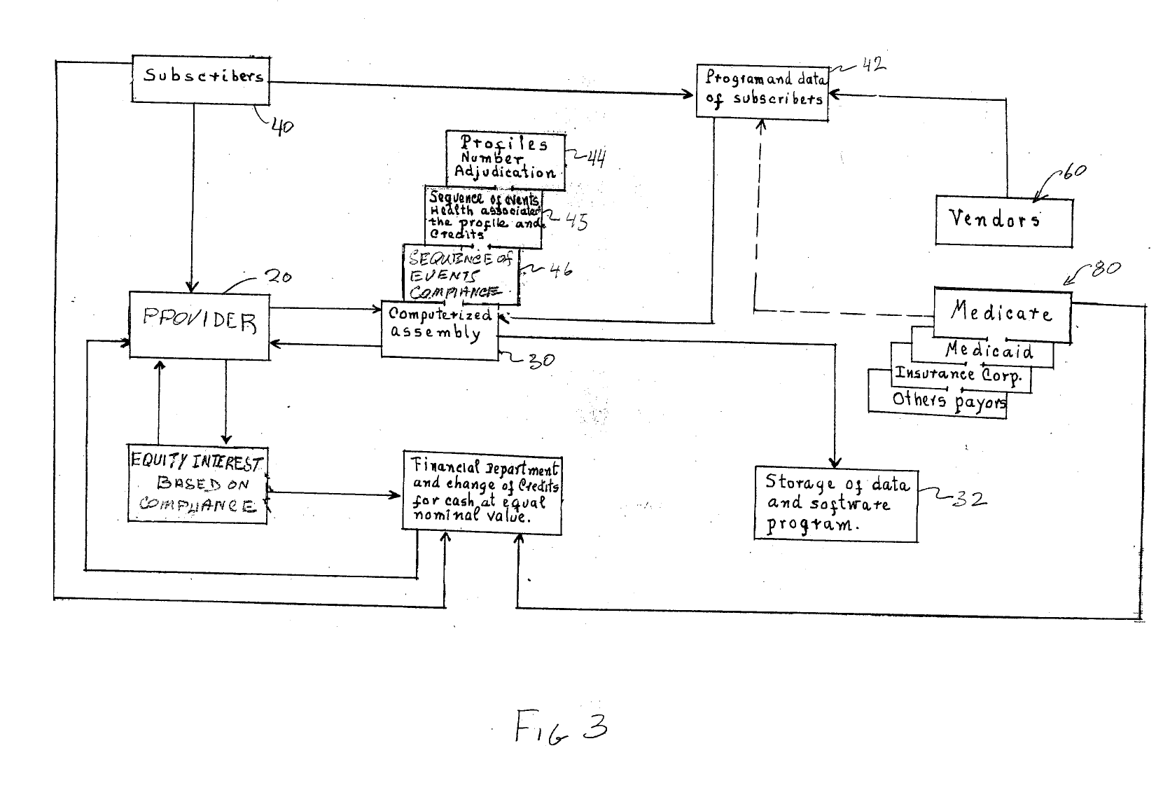

As will also be explained in greater detail hereinafter the health care delivery system and method of the present invention may involve payment for some or all of the medical care treatment, procedures, etc. by payors other than the individual subscribers 40. With reference to FIGS. 1 and 3, such additional or third-party payors may include conventional sources of health care funds such as Medicare, Medicaid, insurance corporations, and/or a variety of other payors.

Upon subscribing to the health care delivery system and method of the present invention, in direct association with a providing entity 20, each of the individual subscribers 40 provides personal particulars and/or characteristics which may include, but are not limited to gender, age, medical and clinical history, etc. In accordance with such personal particulars or characteristics as well as any current or existing medical condition which may require medical attention, the individual subscriber is categorized and assigned at least one of a plurality of predetermined health profiles 44. Each of the plurality of profiles 44 are predetermined and are either specifically or generally defined to include predetermined parameters of personal particulars and/or characteristics which include, but are not limited to, gender, age, medical history, etc. as set forth above. Storage of the plurality of profiles, as defined above, may be maintained within the database and/or software program facilities 32, as also set forth above.

The personal particulars and/or characteristics 42 used to catagorize and/or assign the plurality of profiles 44 are important in the immediate establishing of one or more preventative treatment regimens that can be undertaken by the subscriber in order to minimize or prevent future maladies and/or for the eventual treatment, cure and/or medical procedures rendered to the individual subscribers when it is determined that medical care is necessary. More specifically, when medical attention or care is required to be administered to one of the subscribers, or preventative action is to be initiated, the subscriber's assigned profile 44 is retrieved from the appropriate data base and/or storage facilities 32 and reviewed along with the particular medical condition of the subscriber which requires treatment and/or which is sought to be prevented or minimized. In this regard, it is understood that for the purposes of the present invention, a medical treatment may include steps to cure, prevent, correct and/or minimize an existing condition, and/or preventative steps designated to prevent or minimize the occurrence or severity of a possible future condition. This future condition may include a condition determined as likely or possible based on a subscriber's current health state, age, family history, gender, exposure to other conditions, etc.

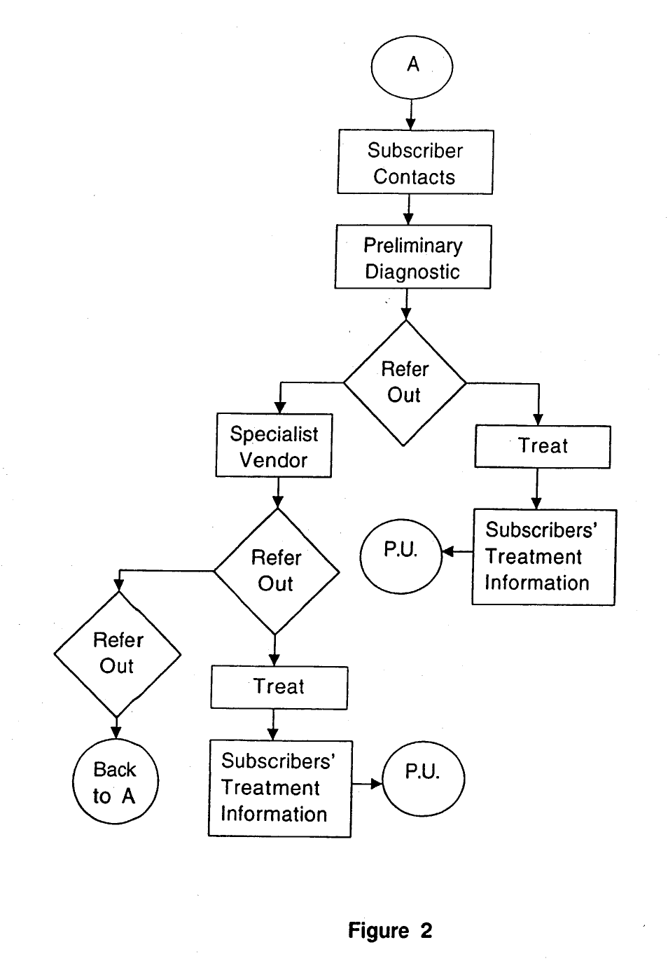

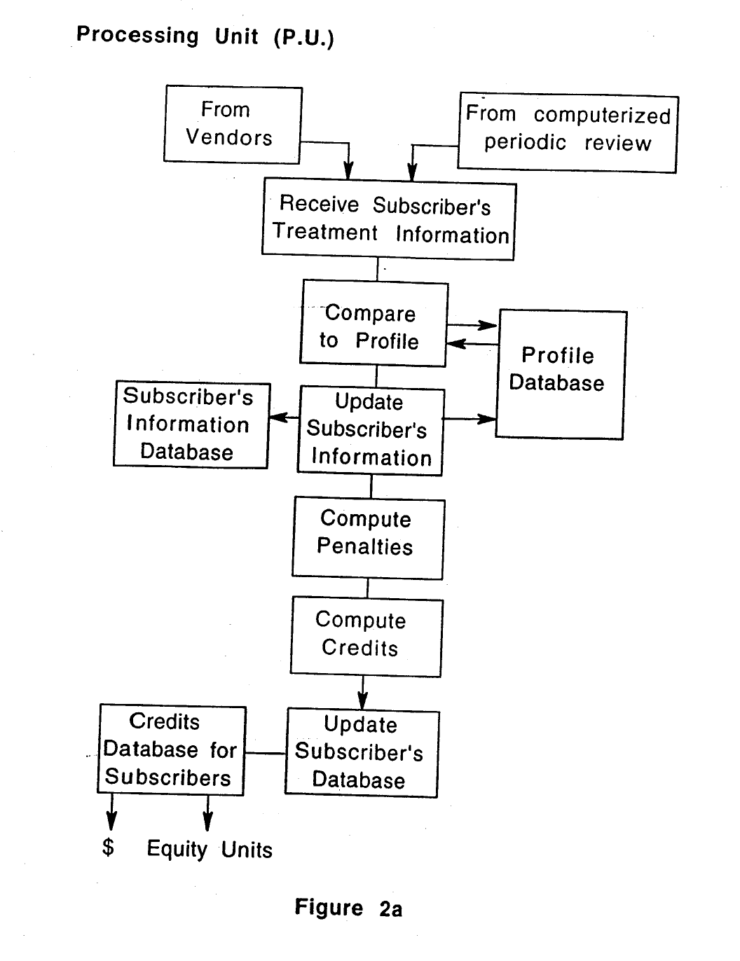

As best shown in FIG. 2, when a subscriber 40 requires medical treatment or care, the providing entity 20 is contacted and the assigned or categorized profile 44 associated with an individual subscriber 40 is reviewed. Thereafter, preliminary diagnostic procedures are conducted and a method of treatment is established. Thereafter, the providing entity 20 determines an individualized health care program which incorporates a treatment regimen for addressing the medical condition of the subscriber 40.

As schematically represented in FIG. 2 the providing entity 20 also must determine whether treatment is to be preformed “in-house” or should best be referred to one or a plurality of health care specialists “vendor”. Assuming the subscriber 40 is to be treated on an in-house basis, the aforementioned treatment regimen will involve specific subscriber's treatment information. The subscriber's treatment information outlines in detail the various treatment, procedures, medications, etc. required for the proper treatment of the individual medical condition for which the individual subscriber 40 requests health care. Subscribers treatment information, as part of the individualized treatment regimen, will be delivered to the processing facilities 30 and or 32 for processing and administration, as described in greater detail in FIG. 2A.

However, the prescribed treatment regimen of a given subscriber 40 may be referred out to a specialist vendor 60, already associated with the providing entity 20 as set forth above. The individual specialist and/or a variety of different health care professionals are consulted to assure that treatment can and will be preformed in accordance with the subscribers treatment information or treatment regimen as dictated by the predetermined health care program, as set forth above. Again it is to be emphasized that the health care program which is determinative of and includes a treatment regimen comprising medication, exercise, outside activities, etc. and define what may referred to as a “sequence of events” 45 which must be preformed on or by the individual subscribers 40. In the context of FIGS. 2 and 3, such “sequence of events” 45 is to be included with and or used synonymously with the subscribers treatment information. As with an in-house treatment regimen described above, the “sequence of events” or subscribers treatment information is properly inputted into the processing facilities and/or storage, data base, etc. 30 and 32.

It is of course well excepted in the medical profession that in order to accomplish a rapid and effective cure or otherwise perfect or maximize the benefit of a medical treatment, including preventative treatments, to an individual, the predetermined procedures or “sequence of events” 45 performed on or by the subscriber must be closely adhered to. Deviation from the sequence of events 45 of the prescribed treatment regimen may in fact delay or prevent cure of a medical condition or result in a worsening thereof dependant upon the degree of compliance, or may result in a failure to receive the benefit sought from a preventative plan. Therefore the health care delivery system and method of the present invention incorporates a motivational feature associated therewith. Such motivational feature comprises a detailed review, over a period of time, of the activities of the subscriber 40 at least to the extent of whether the subscriber has or is complying as at 46 with the sequence of events 45 included in the subscriber's treatment information.

With reference to FIGS. 2A and 3, the compliance 46 of an individual subscriber 40 with the sequence of events 45 of the prescribed treatment regimen is determined, at least in part, from retrieving the stored data regarding the subscribers activities. As indicated with reference to FIG. 2 the subscribers performance is continuously updated by the vendors 60 or in-house personnel, dependent those responsible for the subscriber's care. Such updated information is maintained in the computer, software and/or processor facilities 30 and 32 as part of the treatment information. Accordingly the providing entity has direct access to the compliance performance with the prescribed sequence of events required for medical care, treatment or cure or a diagnosed medical condition. Therefore, the input from the vendors 60 when the mode of treatment is referred out to health care specialists and/or from a computerized periodic review of the various information inputted into the processing utilities 30 and/or 32 from in-house personnel serves to maintain a record of the performance of an individual subscriber. Further, an individual subscribers information is updated based upon both current and past activities relating to the prescribed sequence of events.

The motivational factors included in the health care delivery system and method of the present invention thereby comprises the awarding of both “credits” and/or “penalties” based upon the subscriber being in compliance 46 with the aforementioned sequence of events 45 of the treatment regimen of the health care program assigned to an individual subscriber. Clearly, compliance of a subscriber with the prescribed sequence of events will result in the awarding of credits. To the contrary non-compliance with the sequence of events will result in the awarding of penalties. The motivational factor therefore comes into play when a sufficient amount of credits, in excess of penalties, have been awarded to a subscriber resulting in the granting of motivational rewards to a subscriber 40.

As schematically represented in FIGS. 2A and 3, the motivational awards, when the amount of credits awarded to a subscriber for compliance with the sequence of events is sufficient may include valuable consideration including money and/or an equity interest in a given entity, particularly an equity interest in the providing entity 20. Administration of the granting of motivational awards, either in the form of money, equity interest in the providing entity 20 or other valuable consideration is processed by an appropriate financial department who are responsible for administering the financial services of the providing entity 20, as indicated.

It is again emphasized with respect to the prescribed sequence of events of a treatment regimen, the deciding entity and/or authorized personal will take into consideration the personal particulars and/or characteristics defining a subscriber's profile, as well as the medical and clinical history, including specific medical or preventative conditions for which medical treatment is being rendered. Equitable considerations will of course have to be injected into the administration and determination of compliance with the sequence of events in accordance with philosophies of each providing entity 20.

Accordingly, motivational features involved in the health care delivery system and method of the present invention will urge the individual subscribers to preform the various sequence of events of the treatment regimen and associated prescribed health care program assigned to an individual subscriber. However, in some cases of severe non-compliance, valuable consideration will not be awarded in any form.

The foregoing description conveys the best understanding of the objectives and advantages of the present invention. Different embodiments may be made of the inventive concept of this invention. It is to be understood that all matter disclosed herein is to be interpreted merely as illustrative, and not in a limiting sense.

What is claimed is:

1. A health care system comprising:
a) a plurality of subscribers,
b) a providing entity including at least one predetermined health care program, said providing entity being organizationally structured to facilitate care of said plurality of subscribers through implementation of said health care program,
c) said health care program including a plurality of profiles each of which is determinative of at least one of a plurality of predetermined treatment regimens, and
d) said health care program including an incentive system including both credits and penalties assigned to said plurality of subscribers dependent on a degree of compliance with a designated one of said predetermined treatment regimens.

2. A health care system as recited in claim 1 wherein said plurality of treatment regimens associated with at least some of said profiles differ from one another.

3. A health care system as recited in claim 2 wherein each of said plurality of treatment regimens comprises a sequence of events assigned to one or more of said plurality of subscribers associated with a given profile.

4. A health care system as recited in claim 3 wherein said sequence of events of each treatment regimen is structured to improve or maintain the health care of each of said plurality of subscribers associated with a given profile.

5. A health care system as recited in claim 3 wherein said sequence of events associated with each of said plurality of treatment regimens are structured to be performed by one or more subscribers assigned to a respective treatment regimen.

6. A health care system as recited in claim 5 wherein one or more of said credits are assigned to respective ones of said plurality of subscribers based on satisfactory performance of a corresponding sequence of events.

7. A health care system as recited in claim 6 wherein one or more of said penalties are assigned to respective ones of said plurality of subscribers based on unsatisfactory performance of a corresponding sequence of events.

8. A health care system as recited in claim 7 wherein said incentive system is determined by a collective consideration of said credits and said penalties.

9. A health care system as recited in claim 5 wherein one or more of said penalties are assigned to respective ones of said plurality of subscribers based on satisfactory performance of a corresponding sequence of events.

10. A health care system as recited in claim 1 wherein said incentive system further comprises an issuance of awards when said degree of compliance results in the assignment of an excess credits over penalties.

11. A health care system as recited in claim 10 wherein said awards comprise valuable consideration.

12. A health care system as recited in claim 11 wherein said valuable consideration comprises equity interest in said providing entity.

13. A health care system as recited in claim 1 wherein said incentive system comprises an issuance of awards defined by an equity interest in said providing entity.

14. A health care system as recited in claim 13 wherein said equity interest defines an award issued to at least one of said plurality of subscribers as a part of said incentive system.

15. A health care system as recited in claim 1 wherein each of said plurality of profiles comprises a plurality of personal characteristics collectively determinative of a corresponding treatment regimen.

16. A health care system as recited in claim 1 wherein said providing entity comprises a computerized data base containing said plurality of profiles, each profile defined by a plurality of personal characteristics predetermined to categorize said plurality of subscribers as to a treatment regimen.

17. A health care system as recited in claim 16 wherein said providing entity further comprises processing facilities structured to compare input of personal characteristics from individual ones of said plurality of subscribers with said plurality of personal characteristics of said plurality of profiles.

18. A health care system as recited in claim 17 wherein said processing facilities are further structured to select at least one treatment regimen dependent on a selected one of said plurality of profiles.

19. A health care system as recited in claim 18 wherein said processing facilities are further structured to evaluate and determine said degree of compliance of a subscriber with said selected treatment regimen.

20. A health care system as recited in claim 1 wherein said health care program is structured to assign at least one of said predetermined treatment regimens to said subscriber immediately upon enrollment by said subscriber and identification of a corresponding one of said profiles.

21. A method of administering health care service by a providing entity to a plurality of subscribers, said method comprising:
maintaining a computerized data base of a plurality of profiles each of which is determinative of a treatment regimen,
categorizing each subscriber desirous of receiving health care service into at least one of said plurality of profiles,
assigning a predetermined treatment regimen to each subscriber dependent on the profile into which the subscriber is categorized,
implementing an incentive system based on compliance of respective ones of the subscribers with the assigned treatment regimen,
applying the incentive program by assigning credits and penalties to the subscriber based on the degree of compliance with the assigned treatment regimen, and
issuing awards to those subscribers having been assigned an excess of credits over penalties.

22. A method as recited in claim 21 comprising defining at least one award to include an equity interest in the providing entity.

23. A method as recited in claim 21 further comprising performing a diagnostic on the subscribers desirous of receiving health care service.

24. A method as recited in claim 23 comprising including results of the diagnostic as subscriber input in the categorizing of the subscribers into the plurality of profiles.

25. A method as recited in claim 23 further comprising determining if the treatment regimen for the subscriber is to be performed by the providing entity or by an outside vendor.

26. A method as recited in claim 25 further comprising collecting performance data regarding compliance with the assigned treatment regimen.

27. A method as recited in claim 26 comprising collecting the performance data from either the providing entity or the outside vendor.

28. A method as recited in claim 27 comprising determining the degree of compliance with the assigned treatment regimen based on the collected performance data.

29. A method as recited in claim 28 comprising assigning credits to subscribers evidencing a satisfactory degree of compliance with the assigned treatment regimen.

30. A method as recited in claim 28 comprising assigning penalties to subscribers evidencing an unsatisfactory degree of compliance with the assigned treatment regimen.

31. A method as recited in claim 21 comprising categorizing said subscriber and assigning a preventative one of said predetermined treatment regimens to said subscriber upon enrollment.
